(19) United States
(12) Patent Application Publication
(10) Pub. No.: US 2026/0335068 A1
(43) Pub. Date: Oct. 29, 2026

(54) OPTICAL LENS MATERIALS WITH MULTI-FUNCTIONAL CROSS-LINKERS

(71) Applicant: Alcon Inc., Fribourg (CH)

(72) Inventors: Thomas Joseph Martin, Mansfield, TX (US); Douglas Schlueter, Azle, TX (US); Christina T. Lollar, Arlington, TX (US); Stephen Q. Zhou, Fort Worth, TX (US)

(21) Appl. No.: 19/660,860

(22) Filed: Apr. 28, 2026

Related U.S. Application Data
(60) Provisional application No. 63/796,618, filed on Apr. 29, 2025.

(51) Int. Cl. A61F 2/16; G02B 1/04

(52) U.S. Cl. CPC A61F 2/16; G02B 1/043

(57) ABSTRACT

In general, embodiments of the present disclosure relate to ophthalmic lenses, such as foldable intraocular lenses (IOLs). In particular, embodiments of the present disclosure relate to a foldable ophthalmic lens having high tensile strength, high elongation, and low hardness. In at least some embodiments, an intraocular lens is provided. The intraocular lens includes at least one copolymer where the intraocular lens has a Shore A hardness of about 65 or less and a stress at break of about 6.0 MPa to about 20 MPa. The at least one copolymer includes a first monomeric subunit including a polymerized acrylate or methacrylate group and a phenyl moiety, a second monomeric subunit different from the first monomeric subunit, the second monomeric subunit including an aliphatic monomer, and a multifunctional acrylate or methacrylate cross-linker subunit.

### INTRODUCTION

Aspects of the present disclosure relate to the field of vision correction and, more specifically, to foldable intraocular lenses.

### BACKGROUND

Intraocular lenses (IOLs) are routinely implanted in patients' eyes during cataract surgery to replace the natural crystalline lens. The optical power of the natural crystalline lens can vary under the influence of the ciliary muscles to provide accommodation for viewing objects at different distances from the eye. The ideal ocular implant or IOL, is optically clear and remains so for a prolonged period of time following implantation. The IOL must be soft enough to be folded and delivered through a small incision, while being flexible and rugged enough to withstand the forces of folding, friction within the insertion cartridge interior, and exiting the nozzle tip during lens insertion. Soft lens materials, which allow for small incision delivery, are often accompanied by low strength. Any small defect in the lens or haptic can localize the stress of folding and create a point of failure. To increase the strength of a material, the amount of a cross-linker may be increased. However, this will lower the extension, making the material less tough, less soft, and more difficult to fold. Lowering the cross-linker amount will make the material softer, but is often accompanied with lower strength and susceptibility to form tears.

Accordingly, there is a need for improved lens formulations that produce lens materials which are foldable, resilient, and less susceptible to tears.

### BRIEF SUMMARY

Embodiments described herein generally relate to ophthalmic lenses, such as foldable intraocular lenses (IOLs). In particular, embodiments of the present disclosure relate to a foldable IOL having high tensile strength, high elongation, and low hardness.

Some embodiments provide an intraocular lens including at least one copolymer where the intraocular lens has a Shore A hardness of about 65 or less and a stress at break of about 6 MPa to about 20 MPa. The at least one copolymer includes a first monomeric subunit including a polymerized acrylate or methacrylate group and a phenyl moiety, a second monomeric subunit different from the first monomeric subunit, the second monomeric subunit including an aliphatic monomer, and a multifunctional acrylate or methacrylate cross-linker subunit.

Some embodiments provide an intraocular lens including at least one copolymer where the intraocular lens has a tear strength of about 1.5 MPa to about 15 MPa. The at least one copolymer includes a first monomeric subunit comprising a polymerized acrylate or methacrylate group and a phenyl moiety, a second monomeric subunit different from the first monomeric subunit, a third monomeric subunit including a hydrophilic monomer, and a cross-linker subunit, the cross-linker subunit is selected from the group consisting of trimethylolpropane triacrylate, trimethylolpropane trimethacrylate, pentaerythritol tetraacrylate, pentaerythritol tetramethacrylate, pentaerythritol triacrylate, pentaerythritol trimethacrylate, and combinations thereof.

Some embodiments provide an intraocular lens including at least one copolymer where the intraocular lens has a Shore A hardness of about 60 or less and a stress at break of about 8 MPa to about 20 MPa. The at least one copolymer includes a first monomeric subunit comprising a polymerized acrylate or methacrylate group, a second monomeric subunit different from the first monomeric subunit, and a multifunctional cross-linker subunit including trimethylolpropane trimethacrylate.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1A depicts a frontal view of an intraocular lens, in accordance with some embodiments described herein.

FIG. 1B depicts a peripheral view of an intraocular lens, in accordance with some embodiments described herein.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

In general, embodiments of the present disclosure relate to ophthalmic lenses, such as foldable intraocular lenses (IOLs). For example, embodiments of the present disclosure relate to a foldable IOL having a high tensile strength of 0.6 MPa or greater, a high elongation of greater than 100%, and a low Shore A hardness of about 70 or less, and methods for forming the same. In at least some embodiments, a multifunctional cross-linker is included in a lens formulation providing an IOL having increased tensile properties, such as strength (stress at break) and toughness (total area under the stress-strain curve, tear strength) with little to no increase in hardness. For example, in at least some embodiments, the IOLs can include a tri- or greater functional cross-linker and have a stress at break of about 6 MPa to about 20 MPa, a toughness of about 3 MPa to about 8 MPa, and a Shore A hardness of about 40 to about 65.

Reference will now be made in detail to the embodiments of the disclosure. It will be apparent to those skilled in the art that various modifications, variations and combinations can be made in the present disclosure without departing from the scope or spirit of the disclosure. For instance, features illustrated or described as part of one embodiment, can be used on another embodiment to yield a still further embodiment. Thus, it is intended that the present disclosure cover such modifications, variations and combinations as come within the scope of the appended claims and their equivalents. Other objects, features and aspects of the present disclosure are disclosed in or are obvious from the following detailed description. It is to be understood by one of ordinary skill in the art that the present discussion is a description of exemplary embodiments only, and is not intended as limiting the broader aspects of the present disclosure.

Unless defined otherwise, all technical and scientific terms used herein have the same meaning as commonly understood by one of ordinary skill in the art to which this disclosure belongs. Generally, the nomenclature used herein and the laboratory procedures are well known and commonly employed in the art. Conventional methods are used for these procedures, such as those provided in the art and various general references. Where a term is provided in the singular, the disclosure also contemplates the plural of that term. The nomenclature used herein and the laboratory procedures described below are those known and commonly employed in the art.

The “anterior surface” of a lens, as used herein, refers to the surface of the lens that faces away from the retina of the user. The anterior surface, may also be referred to as the front curve of the lens.

The “posterior surface” of a lens, as used herein, refers to the surface of the lens that faces towards retina of the user. The rear surface, may also be referred to as the base curve of the lens.

As used in this application, the term “polymerizable composition” refers to a composition which can be cured (i.e., polymerized and/or crosslinked) thermally or actinically (e.g., by actinic radiation) to obtain a crosslinked polymer. Examples of actinic radiation are ultraviolet (UV) irradiation, ionized radiation (e.g., gamma ray or X-ray irradiation), microwave irradiation, and the like.

As used in this application, the term “hydrophilic monomer” refers to a vinylic monomer, which as a homopolymer typically yields a polymer that is water-soluble or can absorb at least 10 percent by weight water.

As used in this application, the term “macromer” refers to a medium and high molecular weight compound or polymer that contains one or more ethylenically unsaturated groups. Medium and high molecular weight typically means average molecular weights greater than 700 Daltons.

As used in this application, the term “cross-linker” refers to a compound having at least two ethylenically unsaturated groups.

As used in this application, the term “photoinitiator” refers to a chemical that initiates radical crosslinking/polymerizing reaction by the use of light.

As used in this application, the term “thermal initiator” refers to a chemical that initiates radical crosslinking/polymerizing reaction by the use of heat energy.

As used in this application, the term “polymer” means a material formed by polymerizing/crosslinking one or more monomers or macromers or prepolymers.

As used in this application, the term “molecular weight” of a polymeric material (including monomeric or macromeric materials) refers to the weight-average molecular weight unless otherwise specifically noted or unless testing conditions indicate otherwise.

As used in this application, the term “dye” means a substance that is soluble in a solvent and that is used to impart color. Dyes are typically transparent or translucent and absorb but do not scatter light.

As used in this application, the term “pigment” means a powdered substance that is suspended in a liquid in which the powdered substance is insoluble. Pigments are used to impart color. Pigments, in general, are more opaque than dyes.

In general, embodiments of the present disclosure relate to foldable hydrophobic IOLs having high tensile strength, high elongation, and low hardness, and methods for forming the same. The cross-linkers of the present disclosure contribute subunits of the IOL for providing the beneficial physical properties in the produced IOLs. Embodiments of the present disclosure provide IOLs including multifunctional cross-linkers that are soft enough to be reliably folded for IOL insertion and strong enough to resist damage.

The term “intraocular lens” and its abbreviation “IOL” are used herein interchangeably to describe lenses that are implanted into the interior of the eye to either replace the eye's natural lens or to otherwise augment vision regardless of whether or not the natural lens is removed. Intracorneal lenses and phakic intraocular lenses are examples of lenses that may be implanted into the eye without removal of the natural lens. In many embodiments, the lens can include a controlled pattern of surface modulations that selectively impart an optical path difference between an inner and an outer portion of the lens's optic such that the lens would provide sharp images for small and large pupil diameters as well as pseudo-accommodation for viewing objects with intermediate pupil diameters.

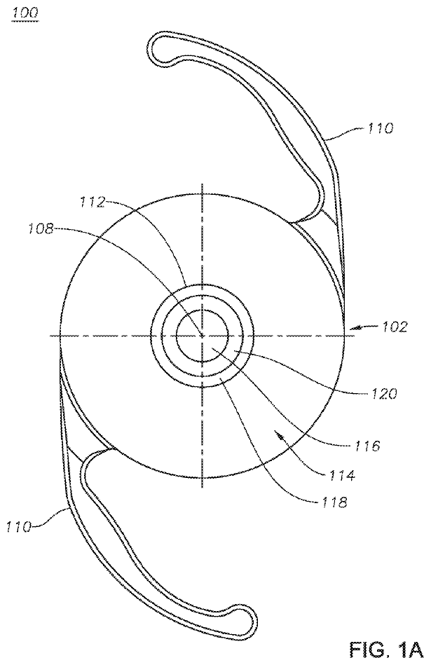

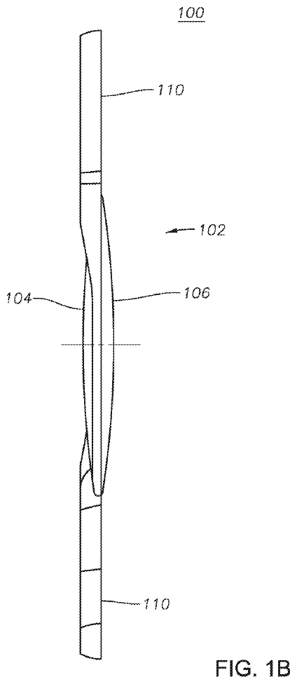

FIGS. 1A-1B are a frontal view and side view, respectively, of an intraocular lens according to some embodiments of the present disclosure. In at least some embodiments, an IOL 100 includes an optic 102 having an anterior surface 104 and a posterior surface 106 that are disposed about an optical axis 108. Optic 102 can have a diameter of about 5 mm to about 10 mm, such as about 6 mm to about 8 mm, such as about 6 mm, about 6.5 mm, or about 7 mm. The IOL 100 may further include a plurality of haptics 110 extending away from the outermost peripheral surface of the optic 102. The haptics 110 can be of any shape, for example, graduated spiral filaments, C-loops (as shown), or flat plate sections, and are used to support the lens within the posterior chamber of the eye. IOLs having any design configuration can be fabricated. In at least some embodiments, the optic 102 and haptic 110 are formed from the same copolymer. In at least some embodiments, the IOL 100 may include other components besides the optic 102 and haptic 110, such other components may be made of the same copolymer as the haptic 110 and optic 102, or another material. In at least some embodiments, the optic 102 and haptic 110 are formed from different copolymers.

In at least some embodiments, the anterior surface 104 of optic 102 includes a first zone 112 extending from the optical axis 108 to a first radial boundary and a second zone 114 extending from the first radial boundary to the edge of the optic 102. Additionally, the first zone 112 may include an inner region 116 and an outer region 118 separated by a phase shift feature 120. The surface features of optic 102 may be configured and/or adjusted to produce an extended depth of focus to meet the needs of the patient. Although the above described first and second zones 112, 114 are depicted and described as being located on anterior surface 104 of optic 102, the present disclosure contemplates that first and second zones 112, 114 may additionally or alternatively be located on posterior surface 106 of optic 102.

In accordance with the disclosure, the IOL 100 includes at least one copolymer that makes up the bulk material of the IOL 100. The copolymer is formed from a polymerizable composition. In at least some embodiments, the copolymer for the IOL 100 generally includes a first monomeric subunit, a second monomeric subunit different from the first monomeric subunit, a third monomeric subunit different from the first monomeric subunit and the second monomeric subunit, and a multifunctional acrylate or methacrylate cross-linker subunit. The polymerizable composition that forms the copolymer may additionally include a chromophore, a blue light absorbing chromophore, a free-radical initiator (photoinitiator or thermal initiator), a visibility tinting agent (e.g., dyes, pigments, or mixtures thereof), antimicrobial agents, bioactive agents, or combinations thereof.

In some embodiments, which can be combined with other embodiments, the copolymer includes about 20 weight percent (wt %) to about 60 wt % of the first monomeric subunit, such as about 30 wt % to about 55 wt %, about 35 wt % to about 50 wt %, about 35 wt % to about 45 wt %, or about 40 wt % to about 45 wt %. In at least some embodiments, the copolymer includes about 20 wt % to about 60 wt % of the second monomeric subunit, such as about 30 wt % to about 55 wt %, about 35 wt % to about 50 wt %, about 35 wt % to about 45 wt %, or about 40 wt % to about 45 wt %. In at least some embodiments, the copolymer includes about 10 wt % to about 25 wt % of the third monomeric subunit, such as about 15 wt % to about 25 wt %, about 15 wt % to about 20 wt %, or about 20 wt % to about 25 wt %. In at least some embodiments, the copolymer includes about 1 wt % to about 10 wt % of the multifunctional acrylate or methacrylate cross-linker subunit, such as about 2 wt % to about 8 wt %, about 2 wt % to about 6 wt %, about 2.5 wt % to about 5 wt %, or about 3 wt % to about 6 wt %. In at least some embodiments, the copolymer may additionally include about 0.5 wt % to about 2 wt % of a chromophore, such as about 1 wt % to about 2 wt %, or about 1 wt % to about 1.5 wt %. In at least some embodiments, the first monomeric subunit may be present in the largest amount as measured by wt % for the copolymer of the three monomeric subunits and multifunctional cross-linker subunit. In at least some embodiments, the second monomeric subunit may be present, as measured by wt % for the copolymer, in the second largest amount of the three monomeric subunits and multifunctional cross-linker subunit, or in a similar amount as the first monomeric subunit. In at least some embodiments, the third monomeric subunit may be present, as measured by wt % for the copolymer, in the third largest amount of the three monomeric subunits and multifunctional cross-linker subunit.

In at least some embodiments, the polymerizable composition that forms the copolymer may additionally include about 0.5 wt % to about 2.5 wt % of a free-radical initiator based on the total weight of the other components of the polymerizable composition, such as about 1 wt % to about 2.5 wt %, about 1 wt % to about 2 wt %, or about 1 wt % to about 1.5 wt %. For example, in at least some embodiments, a copolymer including about 20 wt % to about 60 wt % of the first monomeric subunit, about 20 wt % to about 60 wt % of the second monomeric subunit, about 10 wt % to about 25 wt % of the third monomeric subunit, about 1 wt % to about 10 wt % of the multifunctional acrylate or methacrylate cross-linker subunit, and about 0.5 wt % to about 2 wt % of the chromophore, may be formed from a polymerizable composition including about 20 wt % to about 60 wt % of a first monomer corresponding to the first monomeric subunit, about 20 wt % to about 60 wt % of a second monomer corresponding to the second monomeric subunit, about 10 wt % to about 25 wt % of a third monomer corresponding to the third monomeric subunit, about 1 wt % to about 10 wt % of a cross-linker corresponding to the multifunctional acrylate or methacrylate cross-linker subunit, and about 0.5 wt % to about 2 wt % of the chromophore. The polymerizable composition additionally includes about 0.5 wt % to about 2.5 wt % of a free-radical initiator, where the wt % of the free-radical initiator is based on the total weight of the previously listed components.

In at least some embodiments, the IOL 100 may have a refractive index (RI) of about 1 or greater, such as about 1.5 or greater. In at least some embodiments, the IOL 100 may have a glass transition temperature (T_{g}) of about 3 °C to about 10 °C, such as about 3 °C to about 8 °C, or about 3 °C to about 6 °C. In at least some embodiments, the IOL 100 may have a stress at break of about 6.0 MPa or greater at room temperature (e.g., about 22 °C to about 23 °C), such as about 8.0 MPa or greater, about 10.0 MPa or greater, about 6.0 MPa to about 20 MPa, about 8 MPa to about 20 MPa about 6.0 MPa to about 12 MPa, about 6 MPa to about 10.5 MPa, about 6 MPa to about 10 MPa, about 7 MPa to about 10 MPa, or about 8 MPa to about 10.5 MPa. In at least some embodiments, the IOL 100 may have a percent strain at break of about 100 or greater at room temperature, such as about 105 to about 200, about 105 to about 140, about 110 to about 135, about 110 to about 130, about 110 to about 120, or about 120 to about 140. In at least some embodiments, the IOL 100 may have a toughness of about 3 MPa or greater at room temperature, such as about 3 MPa to about 20 MPa, about 3 MPa to about 8 MPa, about 3.5 MPa to about 7.5 MPa, about 4 MPa to about 7 MPa, about 5 MPa to about 8 MPa, or about 3 MPa to about 5 MPa. The toughness is the total area under the stress-strain curve (AUC) produced when measuring the tear strength. In at least some embodiments, the IOL 100 may have a tear strength of about 1.5 MPa or greater at room temperature, such as about 1.5 MPa to about 15 MPa, about 1.5 MPa to about 5 MPa, about 1.5 MPa to about 4 MPa, about 1.5 MPa to about 3 MPa, about 1.5 MPa to about 2 MPa, about 2.5 MPa to about 3 MPa, or about 2 MPa to about 5 MPa. In at least some embodiments, the IOL 100 may have a Shore A hardness of about 70 or less at room temperature, such as about 65 or less, about 60 or less, about 55 or less, about 40 to about 65, about 50 to about 65, about 55 to about 60, or about 40 to about 50. In at least some embodiments, the IOL 100 may have a dynamic mechanical analysis (DMA) storage modulus of about 5 to about 25, such as about 5 to about 15, about 5.5 to about 14.5, about 6 to about 13, or about 6 to about 10, when measured at 1 Hz and 25 °C. In at least some embodiments, the IOL 100 may have a DMA loss modulus of about 4 to about 25, such as about 4 to about 15, about 4.5 to about 13, about 5 to about 12, or about 5 to about 10, when measured at 1 Hz and 25 °C.

The cross-linkers of the present disclosure contribute to providing beneficial physical properties in the IOLs produced in accordance with the disclosure. The multifunctional acrylate or methacrylate cross-linker includes three or more acrylate or methacrylate groups, such as four or more, or five or more. In at least some embodiments, the multifunctional acrylate or methacrylate cross-linker is an aliphatic cross-linker.

The IOLs disclosed herein show improved tensile properties, such as strength (stress at break) and toughness with little to no increase in hardness. For example, comparative IOLs formed using a cross-linker having two acrylate or methacrylate groups have a stress at break of about 3.5 MPa to about 7 MPa, a toughness of about 1.5 MPa to about 4 MPa, and a Shore A hardness of about 50 to about 65. IOLs formed using a cross-linker having three or more acrylate or methacrylate groups can have, for example, a stress at break of about 6.0 MPa to about 12 MPa, a toughness of 3 MPa to about 8 MPa, and a Shore A hardness of about 40 to about 65. The increased tensile properties with little to no increase in hardness advantageously provides soft material IOLs that may be folded small enough to be inserted through an about 2.4 mm to about 2.8 mm incision in the eye and withstand the forces of folding, friction within the insertion cartage interior (reducing or avoiding IOL defects), and exiting the nozzle tip during lens insertion without damage to the physical or optical properties of the IOL.

In at least some embodiments, the first monomeric subunit is an aromatic monomer including a polymerized acrylate or methacrylate group and a phenyl moiety. Examples of aromatic monomers include 2-phenethyl acrylate, 2-phenethyl methacrylate, benzyl acrylate, benzyl methacrylate, 3-phenylpropyl acrylate, 3-phenylpropyl methacrylate, 4-phenylbutyl acrylate, 4-phenylbutyl methacrylate, 2-phenoxyethyl acrylate, 2-phenoxyethyl methacrylate, or combinations thereof. In at least some embodiments, the first monomeric subunit is provided by 2-phenylethyl acrylate.

In at least some embodiments, the second monomeric subunit is an aliphatic monomeric subunit. Examples of aliphatic monomers include ethyl acrylate, n-propyl acrylate, isopropyl acrylate, n-butyl acrylate, sec-butyl acrylate, n-pentyl acrylate, 3-pentyl acrylate, neopentyl acrylate, n-hexyl acrylate, 2-ethylhexyl acrylate, n-octyl acrylate, methylacrylate, methylmethacrylate, ethylmethacrylate, propylmethacrylate, cyclohexyl acrylate, cyclohexylmethyl acrylate, 2-cyclohexylethyl acrylate, 3-cyclohexylpropyl acrylate, cyclopentyl acrylate, cyclopentylmethyl acrylate, 2-cyclopentylethyl acrylate, 3-cyclopentylpropyl acrylate, isobornyl methacrylate, perfluorohexylethyl-thio-carbonylaminoethylmethacrylate, trifluoroethyl methacrylate, hexafluoro-isopropyl methacrylate, hexafluorobutyl methacrylate, or combinations thereof. In at least some embodiments, the second monomeric subunit includes cyclohexyl acrylate, cyclohexylmethyl acrylate, 2-cyclohexylethyl acrylate, 3-cyclohexylpropyl acrylate, 3-cyclohexylpropyl methacrylate, cyclopentyl acrylate, cyclopentylmethyl acrylate, 2-cyclopentylethyl acrylate, 2-cyclopentylethyl methacrylate, 3-cyclopentylpropyl acrylate, 3-cyclopentylpropyl methacrylate, or combinations thereof. In at least some embodiments, the second monomeric subunit includes a polymerized acrylate or methacrylate group and a cyclic moiety. In at least some embodiments, the second monomeric subunit is provided by 2-cyclohexylethyl acrylate. In at least some embodiments, the second monomeric subunit may have a structure similar to the aromatic structure of the first monomeric subunit, but lack aromaticity.

In at least some embodiments, the third monomeric subunit is a hydrophilic monomeric subunit. Examples of hydrophilic monomers are N,N-dimethylacrylamide (DMA), N,N-dimethylmethacrylamide (DMMA), 2-acrylamidoglycolic acid, 3-acryloylamino-1-propanol, N-hydroxyethyl acrylamide, N-[tris(hydroxymethyl)methyl]-acrylamide, N-methyl-3-methylene-2-pyrrolidone, 1-ethyl-3-methylene-2-pyrrolidone, 1-methyl-5-methylene-2-pyrrolidone, 1-ethyl-5-methylene-2-pyrrolidone, 5-methyl-3-methylene-2-pyrrolidone, 5-ethyl-3-methylene-2-pyrrolidone, 1-n-propyl-3-methylene-2-pyrrolidone, 1-n-propyl-5-methylene-2-pyrrolidone, 1-isopropyl-3-methylene-2-pyrrolidone, 1-isopropyl-5-methylene-2-pyrrolidone, 1-n-butyl-3-methylene-2-pyrrolidone, 1-tert-butyl-3-methylene-2-pyrrolidone, 2-hydroxyethyl methacrylate (HEMA), 2-hydroxyethyl acrylate (HEA), hydroxypropyl acrylate, hydroxypropyl methacrylate (HPMA), 3-hydroxypropyl acrylate, 3-hydroxypropyl methacrylate, trimethylammonium 2-hydroxypropyl methacrylate hydrochloride, 2-hydroxypropyl methacrylate, 2-hydroxypropyl acrylate, 4-hydroxybutyl methacrylate, 4-hydroxybutyl acrylate, aminopropyl methacrylate hydrochloride, dimethylaminoethyl methacrylate (DMAEMA), glycerol methacrylate (GMA), glycerol acrylate, N-vinyl-2-pyrrolidone (NVP), allyl alcohol, vinylpyridine, a C₁-C₄-alkoxy polyethylene glycol (meth)acrylate having a weight average molecular weight of up to 1500, 2-(2-hydroxyethoxy)ethyl acrylate, 2-(2-hydroxyethoxy)ethyl methacrylate, methacrylic acid, N-vinyl formamide, N-vinyl acetamide, N-vinyl isopropylamide, N-vinyl-N-methyl acetamide, allyl alcohol, N-vinyl caprolactam, or combinations thereof. In at least some embodiments, the third monomeric subunit includes HEMA, 3-hydroxypropyl acrylate, 3-hydroxypropyl methacrylate, 4-hydroxybutyl methacrylate, 4-hydroxybutyl acrylate, glycerol acrylate, glycerol methacrylate, N-vinyl pyrrolidinone, poly(ethylene glycol)methyl ether acrylate, poly(ethylene glycol)methyl ether methacrylate, 2-(2-hydroxyethoxy)ethyl acrylate, 2-(2-hydroxyethoxy)ethyl methacrylate, or combinations thereof.

The multifunctional acrylate or methacrylate cross-linker may include trimethylolpropane triacrylate, trimethylolpropane trimethacrylate, pentaerythritol tetraacrylate, pentaerythritol tetramethacrylate, pentaerythritol triacrylate, pentaerythritol trimethacrylate, or combinations thereof. In at least some embodiments, a cross-linker including two acrylate or methacrylate groups may be used. For example, the cross-linker may include 1,4-butanediol diacrylate, 1,4-butanediol dimethacrylate, or combinations thereof.

In at least some embodiments, the polymerizable composition used to form the copolymer includes a chromophore (e.g., as a chromophore unit). In at least some embodiments, the chromophore is a UV-absorbing agent. In at least some embodiments, the chromophore is a polymerizable UV-absorbing agent. Any suitable chromophore may be used. For example, a chromophore may include a benzotriazole-moiety or a benzophenone-moiety. Examples of chromophores include 2-(2-hydroxy-5-vinylphenyl)-2H-benzotriazole, 2-(2-hydroxy-5-acrylyloxyphenyl)-2H-benzotriazole, 2-(2′-hydroxy-3-methacrylamidomethyl-5-tertoctylphenyl)benzotriazole, 2-(2′-hydroxy-5′-methacrylamidophenyl)-5-chlorobenzotriazole, 2-(2′-hydroxy-5′-methacrylamidophenyl)-5-methoxybenzotriazole, 2-(2′-hydroxy-5′-methacryloxypropyl-3′-t-butylphenyl)-5-chlorobenzotriazole, 2-(2′-hydroxy-5′-methacryloxyethylphenyl)benzotriazole, 2-(2′-hydroxy-5′-methacryloxypropylphenyl)benzotriazole, 2-hydroxy-4-acryloxy alkoxy benzophenone, 2-hydroxy-4-methacryloxy alkoxy benzophenone, allyl-2-hydroxybenzophenone, 2-hydroxy-4-methacryloxy benzophenone, 2-(2H-1,2,3-benzotriazol-2-yl)-4-methyl-6-(2-methyl-2-propen-1-yl) phenol (OMTP), 3-(3-(tert-butyl)-5-(5-chloro-2H-benzo[d][1,2,3]triazol-2-yl)-4-hydroxyphenoxy) propyl methacrylate (UV28), 2-tert-butyl-6-(5-chlorobenzotriazol-2-yl)-4-ethenylphenol (UVAM), or combinations thereof. In at least some embodiments, the chromophore includes 2-(2H-1,2,3-benzotriazol-2-yl)-4-methyl-6-(2-methyl-2-propen-1-yl) phenol (OMTP), 3-(3-(tert-butyl)-5-(5-chloro-2H-benzo[d][1,2,3]triazol-2-yl)-4-hydroxyphenoxy) propyl methacrylate (UV28), 2-tert-butyl-6-(5-chlorobenzotriazol-2-yl)-4-ethenylphenol (UVAM), or combinations thereof.

In at least some embodiments, the free-radical initiator is a thermal initiator. Examples of suitable thermal initiators that may be used in polymerizable composition used to form the copolymer include 2,2′-azobis(2,4-dimethylpentanenitrile), 2,2′-azobis(2-methylpropanenitrile), 2,2′-azobis(2-methylbutanenitrile), 2,2′-azobis(isobutyronitrile) (AIBN), peroxides such as benzoyl peroxide, di(4-tert-butylcyclohexyl) peroxydicarbonate (Perkadox® 16), 1,1,3,3-tetramethylbutyl peroxyneodecanoate (Trigonox® 423), tert-butyl peroxybenzoate, di(2-ethylhexyl) peroxydicarbonate, the like, or combinations thereof. For example, in at least one embodiment the thermal initiator is di(4-tert-butylcyclohexyl) peroxydicarbonate.

Suitable photoinitiators that may be used in the polymerizable composition are benzoin methyl ether, diethoxyacetophenone, a benzoylphosphine oxide, 1-hydroxycyclohexyl phenyl ketone and Darocur and Irgacure types, such as Darocur 1173® and Irgacure 2959®. Examples of benzoylphosphine initiators include 2,4,6-trimethylbenzoyldiphenylophosphine oxide; bis-(2,6-dichlorobenzoyl)-4-N-propylphenylphosphine oxide; and bis-(2,6-dichlorobenzoyl)-4-N-butylphenylphosphine oxide. Reactive photoinitiators, which can be incorporated, for example, into a macromer or can be used as a monomer are also suitable.

In accordance with the disclosure, the polymerizable composition of the copolymer is a fluid composition, which can be a solventless blend (e.g., a fluid composition substantially or entirely free of non-reactive diluent-solvent). The polymerizable composition can be prepared by mixing all polymerizable components and other necessary component(s). In at least some embodiments, the polymerizable composition includes, at least, a first monomer corresponding to the first monomeric subunit, a second monomer corresponding to the second monomeric subunit, a third monomer corresponding to the third monomeric subunit, a multifunctional acrylate or methacrylate cross-linker corresponding to the cross-linker subunit, and a free-radical initiator. In some embodiments, the polymerizable composition may further include a chromophore. A solventless polymerizable composition may comprise at least one blending monomer as a reactive solvent for dissolving all other polymerizable components of the solventless polymerizable composition. In at least some embodiments, the first monomer and/or the second monomer is used as a blending monomer in preparing a solventless polymerizable composition.

In at least some embodiments, the polymerizable composition is filtered before being dispensed into a lens mold. The polymerizable composition may be successively filtered through a 2.7 micron, 1.0 micron, and 0.4 micron PTFE (PolyTetraFluoroEthylene) membrane.

Any lens mold for making IOLs, such as the IOL 100, may be used, for example, lens molds employed in cast molding or spin casting. For example, a mold (for cast molding) generally comprises at least two mold sections (or portions) or mold halves, i.e., first and second mold halves. The first mold half defines a first molding (or optical) surface and the second mold half defines a second molding (or optical) surface. The first and second mold halves are configured to receive each other such that a lens-forming cavity is formed between the first molding surface and the second molding surface. The molding surface of a mold half is the cavity-forming surface of the mold and in direct contact with the polymerizable composition.

The mold halves can be formed through various techniques, such as injection molding. The process of the present disclosure is not limited to any particular method of forming a mold. In fact, any method of forming a mold can be used in the present disclosure. The mold halves can be formed through various techniques, such as injection molding or lathing.

Virtually any suitable material for making molds can be used to make molds for making intraocular lenses. For example, polymeric materials, such as polyethylene, polypropylene, polymethylpentene, Topas® COC, or the like can be used. Other materials that allow UV light transmission could be used, such as quartz glass and sapphire.

In accordance with the disclosure, a polymerizable composition can be introduced (dispensed) into a cavity formed by a mold according to any known methods.

After the polymerizable composition is dispensed into the mold, the polymerizable composition is polymerized to produce an IOL. Polymerization may be initiated thermally or actinically to polymerize and crosslink the polymerizable components in the polymerizable composition.

The thermal polymerization is carried out conveniently, for example, at a temperature of about 85 °C to about 110 °C and such as about 90 °C to about 100 °C. The reaction time may vary within wide limits, for example, from about 1 hour to about 4 hours, such as about 3 hours. It is advantageous to previously degas the components used in the polymerization reaction and to carry out said copolymerization reaction under an inert atmosphere, for example under a nitrogen or argon atmosphere.

The actinic polymerization can then be triggered by actinic radiation, for example, a light of a suitable wavelength.

After curing, the steps of opening a mold (i.e., separating the male mold half from the female mold half with the IOL attached onto one of the male and female mold halves) and delensing (i.e., removing the IOL from the lens adhered mold half) are carried out according to any suitable techniques.

In at least some embodiments, the IOL may be cut, for example, by a lathing and/or milling into the desired shape. In at least some embodiments, the IOL is milled before being removed from the mold. In at least some embodiments, the plurality of haptics 110 may be milled.

After the molded IOL is delensed, the IOL may be extracted with an extraction medium. The extraction liquid medium is any solvent capable of dissolving any diluent(s), unpolymerized polymerizable materials, and oligomers in the lens precursor. Water, any organic solvents, or a mixtures thereof can be used to extract the lens. The extracted IOL can be further dried and subject to surface treatment, such as a plasma treatment.

The above disclosure will enable one having ordinary skill in the art to practice embodiments. In order to better enable the reader to understand specific embodiments and the advantages thereof, the following examples are provided. The percentages in the formulations are based on weight percentages unless otherwise specified.

### EXAMPLES

### Example 1

### Effect of Cross-Linker Composition

Polymerizable compositions having different cross-linkers were prepared according to Table 1. Each polymerizable composition included an additional about 1.0 wt % to about 1.8 wt % of the thermal initiator di(4-tert-butylcyclohexyl) peroxydicarbonate (Perkadox® 16), the wt % was calculated based on the weight of the monomer formulation. Prior to use, the inhibitor was removed from all monomers except HEMA by passing the monomer through a column of basic alumina. Low inhibitor grade HEMA was used as received for all experiments. Monomers, cross-linker, chromophore (UV absorber), initiator, and optionally blue light absorbing chromophore were combined in an amber glass bottle. The bottle was sealed and placed on a shaker table until all solids were completely dissolved. The resulting formulation mixture was filtered successively through 2.7 micron, 1.0 micron, and 0.4 micron PTFE membrane filters and dispensed into a polypropylene lens mold. The filled molds were then placed in a programmable convection oven and cured at 95 °C for 3 hours. Once complete, the lens molds were allowed to cool to room temperature. The molds were disassembled and the prescribed IOL shape was milled and the lens removed from the mold. The lenses were solvent extracted in acetone, air dried for at least 2 hours, and vacuum dried at 80 °C for at least 16 hours. Lenses were then exposed to argon vacuum plasma for 5 min.

TABLE 1

| | | | | | | | |
|---|---|---|---|---|---|---|---|
| Components | IOL 1 | IOL 2 | IOL 3 | IOL 4 | IOL 5 | IOL 5 | IOL 7 |

Components

| | | | | | | | |
|---|---|---|---|---|---|---|---|
| 2-phenethyl acrylate | 80.0 | 40.0 | 40.0 | 40.0 | 40.0 | 40.0 | 40.0 |
| 2-cyclohexylethyl | | 36.4 | 35.9 | 36.3 | 35.9 | 35.9 | 35.5 |
| acrylate | | | | | | | |
| HEMA | 15.0 | 20.0 | 20.0 | 20.0 | 20.0 | 20.0 | 20.0 |
| 1,4-Butanediol | 3.2 | 3.2 | 3.2 | | | | |
| diacrylate | | | | | | | |
| pentaerythritol | | | | 2.8 | | | |
| tetraacrylate | | | | | | | |
| pentaerythritol | | | | | 3.2 | | |
| triacrylate | | | | | | | |
| trimethylolpropane | | | | | | 3.2 | |
| triacrylate (TMPTA) | | | | | | | |
| trimethylolpropane | | | | | | | 3.6 |
| trimethacrylate | | | | | | | |
| (TMPTMA) | | | | | | | |
| Chromophores | 1.8 | 0.4 | 0.9 | 0.9 | 0.9 | 0.9 | 0.9 |

Properties

| | | | | | | | |
|---|---|---|---|---|---|---|---|
| EWC @ 35 C BSS | 1.5 | 1.9 | — | 2.2 | 1.2 | 1.7 | 1.7 |
| hydrated | | | | | | | |
| RI (@ 35 C BSS | 1.5470 | 1.5180 | — | 1.5210 | 1.5215 | 1.5201 | 1.5212 |
| hydrated | | | | | | | |
| ABBE @ 35 C BSS | 39.5 | 45.0 | — | 44.6 | 43.9 | 45.6 | 43.8 |
| hydrated | | | | | | | |
| Tg (°C) - mid | 9.1 | 5.4 | 4.6 | 5.1 | 5.2 | 4.2 | 3.6 |
| Stress (MPa) at | 3.8 | 4.8 | 6.5 | 8.0 | 7.7 | 6.7 | 10.0 |
| break @23 C | | | | | | | |
| Strain (%) at | 117 | 118 | 124 | 126 | 106 | 117 | 136 |
| break @23 C | | | | | | | |
| Toughness (MPa), AUC | 2.1 | 2.7 | 3.7 | 5.0 | 4.1 | 3.8 | 7.0 |
| DMA Storage Modulus | 2.8 | — | — | 5.8 | 13.8 | 11.8 | 13.0 |
| (0.1 Hz, 25 C) | | | | | | | |
| DMA Loss Modulus | 2.7 | — | — | 4.5 | 12.3 | 8.6 | 8.3 |
| (0.1 Hz, 25 C) | | | | | | | |
| DMA Tan Delta | 0.98 | — | — | 0.78 | 0.90 | 0.72 | 0.63 |
| (0.1 Hz, 25 C) | | | | | | | |
| Shore A hardness | 56 | 63 | 63 | 57 | 63 | 41 | 56 |
| Tear Strength, | 1.27 | 1.26 | — | 2.03 | 1.74 | 1.86 | 2.58 |
| Average (MPa) | | | | | | | |

IOLs 1-3 were formed using a cross-linker having two acrylate groups. IOLs 4-7 were formed using a multifunctional acrylate or methacrylate cross-linker. IOL 4 was formed using a multifunctional cross-linker having four acrylate groups, and IOLs 5-7 were formed using a multifunctional cross-linker having three acrylate or methacrylate groups. The IOLs formed using the multifunctional acrylate or methacrylate cross-linkers have higher stress at break values (6.7 MPa to 10 MPa) and toughness values (3.8 MPa to 7.0 MPa) than the IOLs formed using a cross-linker having only two acrylate groups (stress at break: 3.8 MPa to 6.5 MPa; toughness: 2.1 MPa to 3.7 MPa), with little to no change in the hardness of the IOLs.

### Example 2

### Refractive Index and Abbe Number

Refractive index and Abbe number were measured using an Abbe refractometer. Samples were equilibrated in balanced salt solution (BSS) at 35 °C prior to measurement. Refractive index measurements were taken at 589 nm (Na light source). The Abbe number (ν_{D}) was calculated using the following formula:

ν_{D}=(n_{D}−1)/(n_{D}−n_{C})

where n_{D}, n_{F}, and n_{C} are the refractive indices of the material at 589 nm (sodium D), 486 nm (hydrogen F), and 656 nm (hydrogen C), respectively

### Example 3

### Differential Scanning Calorimetry (DSC)

The glass transition temperature (T_{g}) was measured by differential scanning calorimetry at 10 °C/min., and was determined as the half-height of the heat capacity increase from the second heating scan.

### Example 4

### Tensile Properties

Tensile properties were determined using dumbbell shaped tension test specimens with a 20 mm total length, length in the grip area of 11 mm, overall width of 2.49 mm, 0.833 mm width of the narrow section, a fillet radius of 8.83 mm, and a thickness of about 0.9 mm. Testing was performed on samples at either 23 °C or 35 °C and 50±10% relative humidity using an Instron tensile testing frame and 50 N load cell. The grip distance was 11 mm and a crosshead speed was 41 mm/minute and the sample was pulled to failure. The strain at break was reported as a fraction of the displacement at failure to the original grip distance. Stress at break was calculated at the maximum load for the sample, typically the load when the sample breaks, also assuming that the initial cross sectional area remains constant. The Young's modulus was calculated from the instantaneous slope of the stress-strain curve in the linear elastic region. The 25% secant modulus was calculated as the slope of a straight line drawn on the stress-strain curve between 0% strain and 25% strain. The 100% secant modulus was calculated as the slope of a straight line drawn on the stress-strain curve between 0% strain and 100% strain.

### Example 5

### Equilibrium Water Content

Equilibrium water content (EWC) was gravimetrically determined using an analytical balance. First, the dry sample weight was obtained, then the sample was equilibrated in balanced salt solution (BSS) at ambient temperature for at least 24 h. The sample was then removed from the BSS, excess surface liquid was removed using a lint free cloth and the sample was weighed. % EWC was determined by the following formula:

$$\% EWC = \frac{( {wt}_{hydrated} - {wt}_{dry} )}{{wt}_{hydrated}} \times 100$$

### Example 6

### Glistening Evaluation

Glistening evaluation was carried out by placing samples in vials containing balanced salt solution (BSS). The vials were equilibrated at 45 °C for at least 20 hours. The vials were then removed and allowed to cool to room temperature for 2 hours. Samples were inspected for glistenings using an optical microscope under dark field conditions with a magnification of at least 10×.

### Example 7

### Storage and Loss Modulus

Storage and loss modulus were determined using a Dynamic Mechanical Analyzer (DMA) in tension mode. Rectangular samples (20×10×0.5 mm) were tested at 1 Hz and 0.5% strain at 25 °C and 35 °C.

### Example 8

### Hardness

Hardness was tested with a Shore A hardness tester at ambient temperature. Films were stacked to create >4 cm high test sample. The probe was depressed against the sample until the reading stabilized. The test was repeated in multiple locations and the values were averaged.

### Example 9

### IOL Delivery Evaluation

Delivery performance of 30D IOLs was evaluated under ambient conditions (20 °C to 22 °C) using a customized delivery device with a force transducer and approximately 1 min dwell time into deionized water maintained at 33 C. Post-delivery evaluations included haptic sticking, optic, haptic and delivery cartridge damage. In general, all the lens optics unfolded quickly and haptics did not stick to the optic region upon delivery. In addition, optics, haptics, and delivery cartridges passed post-delivery cosmetic inspection.

### EMBODIMENTS

The present disclosure is further directed to the following embodiments which may be combined with any embodiments described herein and/or may be isolated (optional) from any embodiments described herein.
Clause 1. An ophthalmic lens comprising:
   at least one copolymer comprising:
      a first monomeric subunit comprising a polymerized acrylate or methacrylate group and a phenyl moiety;
      a second monomeric subunit different from the first monomeric subunit, the second monomeric subunit comprising an aliphatic monomer; and
      a multifunctional acrylate or methacrylate cross-linker subunit; and
   wherein the ophthalmic lens has a Shore A hardness of about 65 or less and a stress at break of about 6 MPa to about 20 MPa.
Clause 2. The ophthalmic lens of Clause 1, wherein the at least one copolymer further comprises a third monomeric subunit different from the first monomeric subunit and the second monomeric subunit.
Clause 3. The ophthalmic lens of Clauses 1 or 2, wherein the third monomeric subunit comprises a hydrophilic monomeric subunit.
Clause 4. The ophthalmic lens of any of Clauses 1 to 3, wherein the at least one copolymer comprises about 20 wt % to about 60 wt % of the first monomeric subunit, about 20 wt % to about 60 wt % of the second monomeric subunit, about 10 wt % to about 25 wt % of the third monomeric subunit, and about 1 wt % to about 10 wt % of the multifunctional acrylate or methacrylate cross-linker subunit.
Clause 5. The ophthalmic lens of any of Clauses 1 to 4, wherein the at least one copolymer comprises about 35 wt % to about 45 wt % of the first monomeric subunit, about 35 wt % to about 45 wt % of the second monomeric subunit, about 15 wt % to about 20 wt % of the third monomeric subunit, and about 2.5 wt % to about 5 wt % of the multifunctional acrylate or methacrylate cross-linker.
Clause 6. The ophthalmic lens of any of Clauses 1 to 5, wherein the second monomeric subunit comprises 2-cyclohexylethyl acrylate.
Clause 7. The ophthalmic lens of any of Clauses 1 to 6, wherein the first monomeric subunit comprises phenethyl acrylate and the second monomeric subunit comprises 2-cyclohexylethyl acrylate.
Clause 8. The ophthalmic lens of any of Clauses 1 to 7, wherein the multifunctional acrylate or methacrylate cross-linker subunit comprises three or more polymerized acrylate or methacrylate groups.
Clause 9. The ophthalmic lens of any of Clauses 1 to 8, wherein the multifunctional acrylate or methacrylate cross-linker subunit is an aliphatic cross-linker subunit.
Clause 10. The ophthalmic lens of any of Clauses 1 to 9, wherein the at least one copolymer further comprises one or more chromophores.
Clause 11. The ophthalmic lens of any of Clauses 1 to 10, wherein the ophthalmic lens has a tear strength of about 1.5 MPa to about 5 MPa.
Clause 12. An ophthalmic lens comprising:
   at least one copolymer comprising:
   a first monomeric subunit comprising a polymerized acrylate or methacrylate group and a phenyl moiety;
   a second monomeric subunit different from the first monomeric subunit;
   a third monomeric subunit comprising a hydrophilic monomer; and
   a cross-linker unit selected from the group consisting of trimethylolpropane triacrylate, trimethylolpropane trimethacrylate, pentaerythritol tetraacrylate, pentaerythritol tetramethacrylate, pentaerythritol triacrylate, pentaerythritol trimethacrylate, and combinations thereof; and wherein the ophthalmic lens has a tear strength of about 1.5 MPa to about 15 MPa.
Clause 13. The ophthalmic lens of Clause 12, wherein the at least one copolymer further comprises one or more chromophore, at least one of the one or more chromophore comprising an ultraviolet (UV)-absorbing agent.
Clause 14. The ophthalmic lens of Clauses 12 or 13, wherein the at least one copolymer comprises about 20 wt % to about 60 wt % of the first monomeric subunit, about 20 wt % to about 60 wt % of the second monomeric subunit, about 10 wt % to about 25 wt % of the third monomeric subunit, and about 1 wt % to about 10 wt % of the cross-linker.
Clause 15. The ophthalmic lens of any of Clauses 12 to 14, wherein the second monomeric subunit comprises a polymerized acrylate or methacrylate group and a cyclic moiety.
Clause 16. The ophthalmic lens of any of Clauses 12 to 15, wherein the ophthalmic lens has a glass transition temperature of about 3 °C to about 10 °C.
Clause 17. The ophthalmic lens of any of Clauses 12 to 16, wherein the ophthalmic lens has a toughness of about 3.5 MPa to about 7.5 MPa.
Clause 18. An ophthalmic lens comprising:
   at least one copolymer comprising:
      a first monomeric subunit comprising a polymerized acrylate or methacrylate group;
      a second monomeric subunit different from the first monomeric subunit; and
      a multifunctional cross-linker comprising trimethylolpropane trimethacrylate; and
   wherein the ophthalmic lens has a Shore A hardness of about 60 or less and a stress at break of about 8 MPa to about 20 MPa.
Clause 19. The ophthalmic lens of Clause 18, wherein the at least one copolymer further comprises a third monomeric subunit different from the first monomeric subunit and the second monomeric subunit, the third monomeric subunit comprising a polymerized acrylate or methacrylate group and hydrophilic monomer.
Clause 20. The ophthalmic lens of Clauses 18 or 19, wherein the ophthalmic lens has a tear strength of about 2.5 MPa to about 3 MPa.

Overall, the present disclosure provides ophthalmic lenses having a stress at break of about 6.0 MPa to about 20 MPa, a toughness of about 3 MPa to about 8 MPa, and a Shore A hardness of about 40 to about 65, and methods for forming the same. In at least some embodiments, a multifunctional cross-linker having three or more acrylate or methacrylate groups is included in a lens formulation providing an ophthalmic lens having increased tensile properties, such as strength and toughness with little to no increase in hardness. The increased tensile properties with little to no increase in hardness advantageously provide IOLs that may be folded small enough to be inserted through an about 2.4 mm to about 2.8 mm incision in the eye and withstand the forces of folding, friction within the insertion cartage interior, and exiting the nozzle tip during lens insertion without damage to the physical or optical properties of the IOLs.

Although various embodiments of the disclosure have been described using specific terms, devices, and methods. Such description is for illustrative purposes only. The words used are words of description rather than of limitation. It is to be understood that changes and variations may be made by those skilled in the art without departing from the spirit or scope of the present disclosure, which is set forth in the following claims. In addition, it should be understood that aspects of the various embodiments may be interchanged either in whole or in part. Therefore, the spirit and scope of the appended claims should not be limited to the description of the preferred versions contained therein.

Some implementations and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Some lower limits, upper limits and ranges appear in one or more claims below.

What is claimed is:

1. An ophthalmic lens comprising:
at least one copolymer comprising:
a first monomeric subunit comprising a polymerized acrylate or methacrylate group and a phenyl moiety;
a second monomeric subunit different from the first monomeric subunit, the second monomeric subunit comprising an aliphatic monomer; and
a multifunctional acrylate or methacrylate cross-linker subunit; and
wherein the ophthalmic lens has a Shore A hardness of about 65 or less and a stress at break of about 6 MPa to about 20 MPa.

2. The ophthalmic lens of claim 1, wherein the at least one copolymer further comprises a third monomeric subunit different from the first monomeric subunit and the second monomeric subunit.

3. The ophthalmic lens of claim 2, wherein the third monomeric subunit comprises a hydrophilic monomeric subunit.

4. The ophthalmic lens of claim 2, wherein the at least one copolymer comprises about 20 wt % to about 60 wt % of the first monomeric subunit, about 20 wt % to about 60 wt % of the second monomeric subunit, about 10 wt % to about 25 wt % of the third monomeric subunit, and about 1 wt % to about 10 wt % of the multifunctional acrylate or methacrylate cross-linker subunit.

5. The ophthalmic lens of claim 2, wherein the at least one copolymer comprises about 35 wt % to about 45 wt % of the first monomeric subunit, about 35 wt % to about 45 wt % of the second monomeric subunit, about 15 wt % to about 20 wt % of the third monomeric subunit, and about 2.5 wt % to about 5 wt % of the multifunctional acrylate or methacrylate cross-linker.

6. The ophthalmic lens of claim 1, wherein the second monomeric subunit comprises 2-cyclohexylethyl acrylate.

7. The ophthalmic lens of claim 1, wherein the first monomeric subunit comprises phenethyl acrylate and the second monomeric subunit comprises 2-cyclohexylethyl acrylate.

8. The ophthalmic lens of claim 1, wherein the multifunctional acrylate or methacrylate cross-linker subunit comprises three or more polymerized acrylate or methacrylate groups.

9. The ophthalmic lens of claim 8, wherein the multifunctional acrylate or methacrylate cross-linker subunit is an aliphatic cross-linker subunit.

10. The ophthalmic lens of claim 1, wherein the at least one copolymer further comprises one or more chromophores.

11. The ophthalmic lens of claim 1, wherein the ophthalmic lens has a tear strength of about 1.5 MPa to about 5 MPa.

12. An ophthalmic lens comprising:
at least one copolymer comprising:
a first monomeric subunit comprising a polymerized acrylate or methacrylate group and a phenyl moiety;
a second monomeric subunit different from the first monomeric subunit;
a third monomeric subunit comprising a hydrophilic monomer; and
a cross-linker unit selected from the group consisting of trimethylolpropane triacrylate, trimethylolpropane trimethacrylate, pentaerythritol tetraacrylate, pentaerythritol tetramethacrylate, pentaerythritol triacrylate, pentaerythritol trimethacrylate, and combinations thereof; and
wherein the ophthalmic lens has a tear strength of about 1.5 MPa to about 15 MPa.

13. The ophthalmic lens of claim 12, wherein the at least one copolymer further comprises one or more chromophore, at least one of the one or more chromophore comprising an ultraviolet (UV)-absorbing agent.

14. The ophthalmic lens of claim 12, wherein the at least one copolymer comprises about 20 wt % to about 60 wt % of the first monomeric subunit, about 20 wt % to about 60 wt % of the second monomeric subunit, about 10 wt % to about 25 wt % of the third monomeric subunit, and about 1 wt % to about 10 wt % of the cross-linker.

15. The ophthalmic lens of claim 12, wherein the second monomeric subunit comprises a polymerized acrylate or methacrylate group and a cyclic moiety.

16. The ophthalmic lens of claim 12, wherein the ophthalmic lens has a glass transition temperature of about 3 °C to about 10 °C.

17. The ophthalmic lens of claim 12, wherein the ophthalmic lens has a toughness of about 3.5 MPa to about 7.5 MPa.

18. An ophthalmic lens comprising:
at least one copolymer comprising:
a first monomeric subunit comprising a polymerized acrylate or methacrylate group;
a second monomeric subunit different from the first monomeric subunit; and
a multifunctional cross-linker comprising trimethylolpropane trimethacrylate; and
wherein the ophthalmic lens has a Shore A hardness of about 60 or less and a stress at break of about 8 MPa to about 20 MPa.

19. The ophthalmic lens of claim 18, wherein the at least one copolymer further comprises a third monomeric subunit different from the first monomeric subunit and the second monomeric subunit, the third monomeric subunit comprising a polymerized acrylate or methacrylate group and hydrophilic monomer.

20. The ophthalmic lens of claim 18, wherein the ophthalmic lens has a tear strength of about 2.5 MPa to about 3 MPa.
